(19) United States
(12) Patent Application Publication
(10) Pub. No.: US 2006/0211662 A1
(43) Pub. Date: Sep. 21, 2006

(54) Combination therapy for topical application in the treatment of age-related macular degeneration and ocular hypertension

(76) Inventors: Charles P. Du Mee, Foster City, CA (US); Gene Barnett, San Francisco, CA (US); Michael Coy, Honolulu, HI (US)

Correspondence Address: Reed Smith LLP, Suite 1400, 3110 Fairview Park Drive, Falls Church, VA, 22042, US

(21) Appl. No.: 11/365,872

(22) Filed: Mar. 2, 2006

Related U.S. Application Data
(60) Provisional application No. 60/657,403, filed on Mar. 2, 2005.

(51) Int. Cl. A61K 31/57; A61K 31/56

(52) U.S. Cl. 514/170

(57) ABSTRACT

A topical drug composition for the treatment of age-related maculopathy, age-rlated macular degeneration, ocular hypertension, glaucoma or other eye disease resulting from increased intraocular pressure comprising a solution of anabolic androgenic agent and 17-β-estradiol suspended or dissolved in a suitable ophthalmic vehicle is disclosed. In the preferred embodiment, 17-β-estradiol, or its esters, is in a lipid vehicle or 17-β-estradiol-3-phosphate, or other esters of 17-β-estradiol, is combined with an anabolic androgenic agent in an aqueous vehicle having a pH of between about pH 6 to pH 8. The composition is administered to the eye in an effective amount to maintain physiologic or normal intraocular pressure or to return elevated intraocular pressure to lower levels.

### FIELD OF THE INVENTION

The present invention is concerned with topically-applied pharmaceutical compositions comprising an effective mixture of estrogen analogs, particularly 17-β-estradiol, or its esters, such as 17-β-estradiol-3-phosphate and androgen analogs and their esters and methods for the treatment of age-related maculopathy (ARM), age-related macular degeneration (ARMD), ocular hypertension, glaucoma or other eye disease resulting from increased intraocular pressure.

### BACKGROUND OF THE INVENTION

It has previously been disclosed that women on hormone replacement therapy often experience an improvement in general ocular condition, including a decrease in intraocular pressure (IOP) (Treister, G, and Mannor, S. Intraocular Pressure and Outflow Facility. Effect of estrogen and combined estrogen-progestin treatment in normal human eyes. Arch. Ophthalmol., 83(3): 311-318 (1970)). More recently, a number of studies have shown an improvement of the ocular conditions of women on hormone replacement therapy (HRT) (Pelit, et al. Tear Function Tests and Conjunctival Impression Cytology Before and After Hormone Replacement Therapy in Postmenopausal Women, Eur. J. Ophthalmol. 13(4): 337-342 (2003); Affinito, P., et al. Effects of Hormone Replacement Therapy on Ocular Function in Menopause, Menopause. 10(5): 482-487 (2003); Marcozzi, G., et al. Effect of Hormone Replacement Therapy on Lacrimal Fluid Peroxidase Activity in Women, Maturitas, 45(3): 225-229 (2003); Marcozzi, G., et al. Age-and Gender-Related Differences in Human Lacrimal Fluid Peroxidase Activity, Ophthalmologica 217(4): 294-297 (2003); Okon, A., et al. The influence of the hormonal replacement therapy on the amount and stability of the tear film among peri-and postmenopausal women, Klin Oczna. 103(4-6):177-181 (2001) Polish; Okon, A., et al. The influence of the hormonal replacement therapy on the amount and stability of the tear film among peri-and postmenopausal women, Klin Oczna. 103(4-6):183-186 (2001) Polish; Jensen, A. A., et al. A Survey of Ocular Complaints in Post-Menopausal Women, J. Assoc. Acad. Minor Phys. 11 (2-3): 44-49 (2000); Sator, M. O., et al. Hormone Replacement Therapy and Intraocular Pressure, Maturitas 28(1): 55-58 (1997); Sator, M. O., et al. Reduction of Intraocular Pressure in a Glaucoma Patient Undergoing Hormone Replacement Therapy, Maturitas 29(1): 93-95 (1998); Akramian J., et al. Estrogen therapy in keratoconjunctivitis sicca, Adv Exp Med Biol. 438:1005-1009 (1998); Sorrentino C., et al. Effect of hormone replacement therapy on postmenopausal ocular function Minerva Ginecol. 50(1-2):19-24 (1998) Italian; Wenderlein M., et al. The “dry eye” phenomenon and ovarian function. Study of 700 women pre-and postmenopausal, Zentralbl Gynakol. 118(12):643-649 (1996) German), and more specifically in the reduction in the IOP (9, 10, 14, 15).

Recent clinical data demonstrates that topical administration of estrogen esters provides relief from the signs and symptoms of dry eye syndrome (Sator, M. O., et al. Reduction of Intraocular Pressure in a Glaucoma Patient Undergoing Hormone Replacement Therapy, Maturitas 29(1): 93-95 (1998); Lubkin, V. Topical estradiol effectively treats postmenopausal dry eye, study shows, Ocular Surgery News. Dec. 1, 2001; Lubkin, V., et al. The Treatment of Perimenopausal Dry Eye Syndrome with Topical Estradiol: Invest., Ophthamol. Vis. Sci. (1992); Lang, Y., et al. The Effects of Hormone Replacement Therapy (HRT) on the Human Eye, Harefuah 141(3): 287-291 (2002); Hebrew, Smith W., et al. Gender, oestrogen, hormone replacement and age-related macular degeneration: results from the Blue Mountains Eye Study. Aust N Z J Ophthalmol. 25 Suppl 1:S13-5 (1997)). It has also been shown that one or more members of the class of compounds characterized as anabolic androgens, when instilled in the eye, are effective in lowering elevated intraocular pressure (U.S. Pat. No. 4,617,299).

The systemic treatment of postmenopausal women with estrogen has also been indicated to reduce the incidence, or delay the onset, of ARMD, and estrogen loss during menopause has been suggested to be involved in the dysregulation of molecules that influence the turnover of extra cellular matrix (ECM) in Bruch's membrane, that leads to the buildup of ECM deposits under the retinal pigment epithelium that is characteristic of ARMD. (Rakic, J. M., et al., Estrogens Reduce the Expression of YKL-40 in the Retina: Implications for Eye and Joint Diseases, IOVS, Vo. 44, No. 4, April (2003); Munaut, C., et al., Presence of Oestrogen Receptor Type 0 in Human Retina, Br. J. Opthalmol., 85:877-882 (2001); Cousins, S. W., Female Gender, Estrogen Loss, and Sub-RPE Deposit Formation in Aged Mice, IOVS, Vol. 44, No. 3, March (2003); Hammond, C. B, et al., Consequences of Estrogen Deprivation and the Rationale for Hormone Replacement Therapy, The American Journal of Managed Care, 6(14):S746-S760 (2000); Marin-Castano, M. E., et al., Regulation of Estrogen Receptors and MMP-2 Expression by Estrogens in Human Retinal Pigment Epithelium, IOVS, Vol. 44, No. 1 (2003); Wang, J. J., et al., Bilateral Involvement by Age Related Maculopathy Lesions in a Population, Br. J Ophthalmol., 82;743-747 (1998); Eye Disease Case-Control Study Group. Risk Factors for Neovascular Age-Related Macular Degeneration, Arch. Ophthalmol., 110: 1701-8 (1992); Lang, Y., et al., The Effects of Hormone Replacement Therapy (HRT) on the Human Eye, Harefuah, 141(3): 287-291 (2002) Hebrew.)

Clinical data also demonstrates that topical administration of estrogen esters improves symptoms of dry eye syndrome (Lubkin, V. Topical estradiol effectively treats postmenopausal dry eye, study shows. Ocular Surgery News. Dec. 1, 2001; Lubkin, V., et al. The Treatment of Perimenopausal Dry Eye Syndrome with Topical Estradiol: Invest. Ophthamol. Vis. Sci. (1992); Sator, et al., Treatment of Menopausal Keratoconjunctivitis Sicca with Topical Oestradiol, Br. J. Obstet. Gynaecol., 105(1):100-2 (1998)). It has also been proposed that the topical treatment of androgens may be beneficial to the treatment of inflammation-related forms of dry eye syndrome (Sullivan, D. A. U.S. Pat. No. 6,107,289).

Based on the positive effects on ocular health of systemic hormone replacement therapy, as well as the beneficial effect on dry eye syndrome resulting from the topical administration of estrogen or its esters, and biochemical evidence of the possible benefit of androgens, it is reasonable to assume that topical administration of combinations of estrogen or its esters, and androgens or their esters would have a beneficial effect on treating ARM and ARMD, reducing IOP, as well as on general ocular health. However, until now, useful pharmaceutical compositions for the topical administration of estrogen esters directly to the eyes for the treatment of ARM, ARMD or ocular hypertension have not been proposed.

### SUMMARY OF THE INVENTION

Accordingly, it is a principal objective of this invention to provide methods and pharmaceutical compositions comprising estrogen esters and androgen analogues for the treatment of age-related macular degeneration and ocular hypertension by ophthalmic topical administration to the ocular or conjunctival surface of the eye of an effective amount of a mixture of 17-β-estradiol or its esters or 17-β-estradiol-3-phosphate or its esters and an anabolic androgenic agent or their related derivatives and esters, dissolved in a suitable vehicle, to reduce intraocular pressure. The illustrative vehicle comprises a lipid (oil based) suspension or an aqueous solution having a pH within the range of 4-8, preferably pH 6-8. It is contemplated that this invention can also utilize a liposomal vehicle as well.

More particularly, the object of the invention is to provide specific drug products applicable to these purposes, and the methods of preparation and application of the same. Even more particularly, this invention provides a composition useful for reducing pathological elevated intraocular pressure.

Preferred anabolic androgenic agents for the purposes of this invention include 17-α-methyl-testosterone, oxandrolone, norethandrolone, bolasterone, methandrostenolone, oxymetholone, dihydrotestosterone, their esters, and their cationic or phosphate or phosphorylated derivatives, designed to increase the solubility in hydrophilic media.

It is to be understood that both the foregoing general description and the following detailed description are exemplary and explanatory, and are not intended to limit of the invention as claimed. Other objects and features of the invention will become apparent from the following detailed descriptions. All references cited in the instant disclosure are incorporated herein by reference.

### DETAILED DESCRIPTION OF THE PREFERRED EMBODIMENTS OF THE PRESENT INVENTION

The present invention provides a method for the treatment of ARMD or ocular hypertension by direct application of combination of an estrogen analogue and androgen analogue-containing composition in proximity to the conjunctival surface of the eye. Accordingly, in the method of the invention, the therapeutically active combination is applied locally to the site where it is needed, rather than being systemically delivered throughout the body. This provides numerous advantages, including the flexibility to tailor the dose for maximum effect with reduced concern for triggering unwanted side effects in other parts of the body. Consequently, topical administration, according to the invention, may permit the use of higher localized doses with reduced side effects, which can enhance the effectiveness of the treatment as well as patient compliance.

Similarly, the compositions useful in the invention may contain any therapeutically effective androgen analogue, including esters and salts thereof. In preferred embodiments, the composition comprises an androgen analogue selected from the group consisting of 17-α-methyl-testosterone, oxandrolone, norethandrolone, bolasterone, methandrostenolone, oxymetholone, and dihydrotestosterone, including their esters and their cationic sodium, potassium or phosphate derivatives. Particularly preferred steroids are 17-α-methyltestosterone and oxandrolone.

The estrogen and androgen compositions may be formulated and applied separately to the eye in the method of the present invention, or they may be formulated and applied as a single composition. Preferably, they are formulated and applied as a single composition. The preferred embodiment or formulation comprises a solution, suspension or cream of an estrogen derivative such as 17-β-estradiol (or the 3-phosphate disodium salt) and an anabolic androgenic agent preferably selected from the group consisting of 17-α-methyl-testosterone, oxandrolone, norethandrolone, bolasterone, methandrostenolone, oxymetholone, and dihydrotestosterone, including their esters and their cationic sodium, potassium or phosphate derivatives

The amount of active ingredient that is to be administered depends on the age of the patient, the intraocular pressure response, the particular disease condition to be treated, such as age-related macular degeneration, the frequency of administration, and means of administration, and the particular steroids employed. The concentration of active ingredients can range from about 0.001 percent to about 10 percent by weight in ophthalmic solution or about 0.0001 milligram to about 10 milligram per administration. The most preferred concentration is about 0.004 milligram to about 4.0 milligrams per administration.

The “effective amount” or “pharmacologically effective amount” of active ingredients in a unit dose depends upon a number of factors. Included among those factors are the carrier when used, the tolerance for the active ingredients, the gender of the patient, the response elicited, and the number of unit dose administrations desired to be used.

In this invention, the combination therapy is administered to eyes exhibiting elevated intraocular pressure, usually presenting elevated intraocular pressure of 21 millimeters Hg or greater as measured with standard tonometric techniques such as Schiotz, MacKay Marg or applanation tonometry. Additional criteria for commencing the prescribed therapy are presentation of the standard diagnostic criteria for primary open angle glaucoma, such as glaucomatous field loss or optic nerve head damage.

The preferred estrogen compound is known as 17-β-estradiol 3-phosphate disodium and 1,3,5 (10)-estratriene-3,17 beta-diol 3-phosphate disodium. The formula is C₁₈H₂₃O₅P₁ Na₂, having a molecular weight of 396.3 (anhydrous).

Each gram of 17-β-estradiol (as the 3-phosphate disodium salt) contains approximately 687 milligram of 17-β-estradiol on an anhydrous basis. 17-β-estradiol (as the 3-phosphate disodium salt) is available commercially, such as from Research Plus, Inc., Bayonne, N.J. 07002 (catalog No. 1850-5). The compound is a white crystalline powder with an ill-defined melting point and purity better than 97%. The material is to be stored in sealed vials under refrigeration when not in use.

Regarding the androgenic agent, selection of the most appropriate therapeutic androgen will depend upon a given hormone's activity, potential side effects and form of administration. For example, topical testosterone may be quite effective in reducing lacrimal inflammation, and its methylated analogue appears to have no toxic side effects on parameters such as intraocular pressure (P. A. Knepper, J. A. Collins, and R. Frederick, Effect of Dexamethasone, Progesterone, and Testosterone on IOP and GAGs in the Rabbit Eye, Invest. Ophthalmol. Vis. Sci. 26:1093-1100 (1985)). However, a variety of other modified and/or anabolic androgens (J. D. Wilson and D. W. Foster, eds., Williams Textbook of Endocrinology, W B Saunders Company, Philadelphia (1985); Vida, J. A., “Androgens and Anabolic Agents,” Academic Press, New York (1969)) may be more effective than testosterone. In addition, with regards to administration, if the steroids are to be complexed to a carrier vehicle (e.g., hyaluronate), then a nitrogenated analogue might be indicated or a phosphorylated analogue if an aqueous solution is desired.

Androgens to be used include testosterone, dihydrotestosterone (also termed allodihydrotestosterone, androstanolone, stanolone, 5 alpha-dihydrostestosterone), fluoxymesterone, stanozolol, nortestosterone propionate, dehydroepiandrosterone (an androgen precursor, also termed androstenolone, dehydroisoandro-sterone, DHEA, transdehydroandrosterone), oxandrolone; methyldihydrotestosterone (also termed methylandrostanolone), oxymetholone, 5 alpha-androstan-17β-ol-3-oxime, 5 alpha-androstan-17 alpha-ol-3-one-acetate, (1) 2,(5 alpha)-androsten-17β-ol, 5 alpha-androstan-2 alpha-methyl-17β-ol-3-one, and methyltestosterone, and their derivatives and esters.

These androgenic agents are representative of the major structural subclasses of androgens, as disclosed in Vida (Vida, J. A., “Androgens and Anabolic Agents,” Academic Press, New York (1969)), hereby incorporated by reference. The subclasses include (a) androgenic compounds with unusual structural features (e.g., 17 alpha-methyl-17β-hydroxy-2-oxa-5 alpha-androstan-3-one, also termed oxandrolone); (b) testosterone derivatives (e.g., methyltestosterone); (c) 4,5 alpha-dihydrotestosterone derivatives (oxymetholone); (d) 17β-hydroxy-5 alpha-androstane derivatives containing a ring A unsaturation, excluding testosterone derivatives (e.g., 2,(5 alpha)-androsten-17β-ol); and (e) 19-nortestosterone derivatives (e.g., 19-nortestosterone propionate).

Also, relative to standards (typically testosterone), these androgens include compounds displaying: (a) augmented androgenic (i.e., virilizing) activity coupled with an even larger increase in anabolic activity (e.g., fluoxymesterone); (b) enhanced anabolic action with unchanged androgenic effects (e.g., oxymetholone, dihydrotestosterone); (c) decreased androgenic ability with unchanged anabolic activity (e.g., 19-nortestosterone propionate); and (d) decreased androgenic capacity paralleled by increased anabolic activity (e.g., oxandrolone, stanozolol).

Preferred androgenic agents of this invention are those which have far more anabolic, than virilizing effect, (e.g., oxandrolone possesses 322% of the anabolic and 24% of the androgenic activity of methyltestosterone (Vida, J. A., “Androgens and Anabolic Agents,” Academic Press, New York (1969)).

Further preferred androgenic agents are nitrogen-substituted androgens such as 5 alpha-androstan-17β-ol 3-oxime, which is created by the substitution of a nitrogen derivative for the 3-ketone function in dihydrotestosterone (very potent androgen) (Vida, J. A., “Androgens and Anabolic Agents,” Academic Press, New York (1969)). This substitution does not inhibit androgen activity (Vida, J. A., “Androgens and Anabolic Agents,” Academic Press, New York (1969)) and may permit steroid binding to hyaluronate for topical administration. Of interest, a variety of other nitrogenated androgens have been shown to express increased anabolic, but decreased androgenic, activity. These compounds typically contain 3-substitutions, but not nitrogen incorporation in the steroid ring structure, which appears to abolish androgen action (Vida, J. A., “Androgens and Anabolic Agents,” Academic Press, New York (1969)).

In order to increase the aqueous solubility of the androgenic agents, phoshorylated ester derivatives of the androgens are preferred and can be prepared by means commonly available in the art. For example, the most convenient method of synthesis of steroid esters is reaction of the steroid in a 2:1 mixture of pyridine and the anhydride of the desired ester: for example, propionic anhydride would be used to make the propionate ester. A large excess (at least 10 times) of the anhydride compared to the steroid would be required. This would then be purified by diluting with at least 10 parts of water to each part of pyridine, adding 1 part ether, decanting the water after shaking, and then washing with 10 parts water repeatedly in a separatory funnel. This would be followed preferably by recrystallization or chromatography for purification.

The delivery vehicle for the combination therapy may be supplied as an over-the-counter artificial tear (solution). However, a preferred vehicle has a composition as shown in Table IV. A more preferred composition of the pharmaceutical carrier is: Dibasic sodium phosphate, USP 0.3%; Sodium Chloride, USP about 0.6%; Edetate disodium, USP 0.1%; Povidone K-15 or K17, USP 0.37%; Poloxamer NF, 0.004%, PEG 0.12%; HEC NF, 0.2%, Purified water, USP, q.s to 100%; HCl or NaOH to adjust pH to pH 6-8. The preferred carrier may further comprise one or more preservatives selected from the group consisting of methylparaben (0.005-0.5 w/w %), proplyparaben (0.005-0.5 w/w %), benzalkonium chloride (0.005-1.0%) and phenoxyethanol (0.005-1.0 w/w %). Other modifications of the carrier solution may be done without departing from the scope of the pharmaceutically acceptable carrier of the present invention.

TABLE IV

| Compound | Concentration (w/w %) |
|---|---|
| Povidone (USP) | 0.05-2.0% |
| Hydroxyethylcellulose (USP) | 0.05-1.0% |
| Sodium chloride (USP) | 0.2-0.9% |
| Anhydrous sodium phosphate (Na₂HPO4) | 0.05-1.0% |
| (USP) | |
| Poloxamer NF | 0.001-0.05% |
| Polyethylene glycol | 0.05-1.0% |
| Disodium edetate (USP) | 0.05-1.0% |
| dil. HCl or NaOH for pH adjustment | qs |
| purified water (USP) | qs |

The synthesis and preparation of the androgenic agents of the present invention are well known in the art and typically belong to the major structural subclasses of androgens, as disclosed in Vida, hereby incorporated by reference. Preferred androgenic agents of this invention are those which have more anabolic, than virilizing effect, (e.g., oxandrolone possesses 322% of the anabolic and 24% of the androgenic activity of methyltestosterone (Vida, J. A., “Androgens and Anabolic Agents,” Academic Press, New York (1969))).

In one embodiment, the anabolic androgenic agent or compound is present preferably in a concentration of about 0.001 to about 10 percent by weight of the composition. During treatment, the anabolic androgenic agent is administered to the eye by contacting the affected eye with a dosage in the range of about 0.0001 milligrams to about 10 milligrams per administration, the preferred dosage range being about 0.004 to about 4.0 milligrams per administration. The administrations may be continuous or repeated over a period of time.

In an alternate embodiment, it is contemplated that a sterile, ophthalmic solution of 17-β-estradiol and anabolic androgenic agent can be comprised of a liposomal drug delivery system (Margalit R., “Liposome-Mediated Drug Targeting in Topical and Regional Therapies”, Crit. Rev. Ther. Drug Carrier Syst,. 12(2-3):233-61 (1995)). Liposomal therapy has been successfully used in ophthalmology not only for pre- and postoperative antisepsis, but also for the treatment of bacterial and viral conjunctivitis and for prophylaxis against ophthalmia neonatorum (Reimer K, et al., “Povidone-Iodine Liposomes—An Overview”, Dermatology, 195 Suppl. 2:93-9 (1997)). A method for formulating such a product can be found in U.S. Pat. No. 5,662,931, and herein incorporated by reference.

In yet another embodiment, the composition comprises a sterile, ophthalmic suspension of 17-β-estradiol cypionate dissolved to form a 0.1% (by volume) solution in a vehicle which may in one embodiment take the form of a lipid based solution having a pH within the range of 4-8 with a preferred range of about 6-8.

In yet another embodiment, the composition comprises a sterile, ophthalmic solution of 17-β-estradiol (as 3-phosphate disodium salt) and androgenic agent dissolved to form a 0.1% (by volume) solution in a vehicle which may in one embodiment take the form of an over-the-counter artificial tear solution. The concentration of 17-β-estradiol in the vehicle is increased or decreased depending on the desired activity of the steroids.

Below are alternate embodiments of the drops.

A. An eye-drop composition comprising an anabolic androgenic compound such as 17-α-methyl testosterone (and/or its water-soluble derivatives) and 17-β-estradiol (as the 3-phosphate disodium salt) and its water-soluble, storage-stable derivatives (beta-estradiol glucuronide, beta-estradiol hemisuccinate, beta-estradiol phosphate, beta-estradiol sulfate and their 3,17 diesters, 17 monoesters and 3 monoesters). The 17-β-estradiol 3-phosphate disodium salt is employed in the preferred embodiment because of the enhanced solubility and stability of the particular derivative at essentially neutral pH 6-8 (though could have a pH between 4-8) and the ease of sterile ophthalmic manufacture.

TABLE I
B. The sterile ophthalmic ointment formulated to melt
at body temperature containing:

| Compound | Concentration (w/w %) |
|---|---|
| 17-β-estradiol (microcrystalline) | 0.001-1.0 |
| 17-α-methyl-testosterone | 0.001-1.0 |
| Propyl paraben (USP) | 0.2 |
| Anhydrous liquid lanolin | 5.0 |
| mineral oil (USP) | 10.0 |
| white petrolatum (USP) | 84.6-84.7 |

TABLE II
C. A sterile aqueous ophthalmic suspension and formulated to contain:

| Compound | Concentration (w/w %) |
|---|---|
| 17-β-estradiol phosphate | 0.001-1.0 |
| 17-α-methyl-testosterone phosphate | 0.001-1.0 |
| Sodium chloride (USP) | 0.2-0.9% |
| Anhydrous sodium phosphate (Na₂HPO4) | 0.05-1.0% |
| (USP) | |
| Poloxamer NF | 0.001-0.05% |
| Polyethylene glycol | 0.05-1.0% |
| Disodium edetate (USP) | 0.05-1.0% by volume |
| dil. HCl or NaOH for pH adjustment | qs |
| purified water (USP) | qs |
| Povidone (USP) | 0.05-2.0% |
| Hydroxyethyl Cellulose | 0.05-1.0% |

TABLE III
D. A sterile aqueous ophthalmic suspension and formulated to contain:

| Compound | Concentration (w/w %) |
|---|---|
| 17-β-estradiol 3-phosphate | 0.001-1.0 |
| Androgenic agent (as the phosphoester | 0.001-1.0 |
| derivative) | |
| Polyethylene glycol | 0.12 |
| Povidone (USP) | 0.37 |
| Hydroxyethylcellulose (USP) | 0.2 |
| Sodium chloride (USP) | 0.6 |
| Disodium edetate (USP) | 0.1 |
| Poloxamer NF | 0.004 |
| dil. HCl for pH adjustment | qs |
| purified water (USP) | qs |

The aqueous ophthalmic suspensions may contain one or more preservatives selected from the group consisting of methylparaben (0.005-0.5 w/w %), propylparaben (0.005-0.5 w/w %), benzalkonium chloride (0.005%-1.0%) and phenoxyethanol (0.005-1.0 w/w %).

The following is a description of the manufacturing and packaging procedure for a preferred drug product of our invention. More information on the preparation and characteristics of 17-β-estradiol 3-phosphate is set forth in the article by Diczfalusy (Diczfalusy, E. High Molecular Weight Enzyme Inhibitors, pp. 1675-1689, Chemica Scandinavia Vol. 12 (1958) No. 8.) which is incorporated herein by reference. The method of synthesis of 17-β-estradiol 3-phosphate disodium is briefly described as follows:

17-β-estradiol 17-acetate (Molecular Weight=314.4, Melting Point 220-224° C. and optical rotation 47°) is phosphorylated in the presence of concentrated ortho-phosphoric acid (H₃PO₄) with heat and refluxing to yield the intermediate 17-β-estradiol 3-phosphate 17-acetate. The latter compound is selectively hydrolyzed in the presence of sodium bicarbonate in aqueous alcohol to yield sodium acetate and 17-β-estradiol 3-phosphate disodium. The desired steroid phosphate ester is recrystallized from dilute alcohol.

The preferred drug product used in our invention is manufactured and packaged as follows:
i) A calculated amount of anabolic androgen and 17-β-3-estradiol (as 3-phosphate disodium salt) on an “as is basis” is weighed on a suitable balance and transferred to a sufficient volume of vehicle.
ii) The drug product is mixed until a solution of the steroids in the vehicle is obtained. (The pH of the solution may be adjusted to about pH 7 with dilute hydrochloric acid (HCl) or dilute sodium hydroxide (NaOH) if required). The drug product is brought to final volume with additional vehicle and mixing.
iii) The drug product is sterile filtered using an appropriate sterile filter assembly and a suitable syringe and filled directly into previously sterilized (see iv) 15 ml dropping bottles with a snap-tip dropper insert and polypropylene overcap (Wheaton Scientific, Millville, N.J. 08332). This portion of the operation is performed directly in front of a class 100 environment.
iv) Air blow Wheaton dropping bottles, inserts and caps are placed inside low density polyethylene sterilizing bag and the bag and contents are sterilized in a 3M ETO sterilizer unit for about 2 hours.
Solutions of drug product are preferably stored at controlled room temperature (15 to 30° C.) preferably at 22 to 24° C. as long as adequate physical stability (i.e., clarity of solution) is maintained. Otherwise storage under refrigeration (less than 10° C.) may be required.

It is also contemplated in an alternate embodiment, that the above composition further comprises a preservative system. More particularly, this alternate embodiment can use methylparaben and/or propylparaben at a concentration (w/w) of about 0.01-0.5%, benzalkonium chloride at a concentration of about 0.005-1.0%, and/or phenoxyethanol at a concentration (w/w) of about 0.01-1.0%.

The placebo used in controlled clinical trials is the preferred vehicle used in the manufacture of the drug product, namely an artificial tear-like solution, similar to the formula of which is identified previously. See for example Table III. The placebo is a non-prescription, artificial tear-like product that can be used to provide temporary relief of dry eye symptoms. It contains mucin-like substances, povidone and hydroxyethylcellulose which mimic the action of the conjunctival mucus or render the surface of the eye more wetable. The vehicle helps keep the eye moist and assures that the tear film can spread easily and evenly over the eye surface.

The preferred vehicle for 17-β-estradiol (as 3-phosphate disodium salt) and anabolic androgenic agent (as the phosphate ester) has the following attributes:
a) a sterile, buffered isotonic solution.
b) contains mucin-like substances that tend to increase the contact time between the active drug substance (17-β-estradiol (as the 3-phosphate disodium salt and 17-α-methyl-testosterone as 3-phosphate salt) and the eye surface.
c) free of benzalkonium chloride (if the phosphate esters or salts are used), which is a cationic surfactant that is known to be incompatible in solutions with steroid sodium phosphate salts.

The following quality control procedures are employed to assure identity, strength, quality and purity of the drug product:

Representative samples of finished drug product are opened and examined for clarity of solution (clear, colorless to pale yellow solution, essentially free of foreign matter), pH content (not less than 6 and not more than 8) and a simple potency assay (absorbance read at 280 nanometers using 1 centimeter cells in a suitable spectrophotometer after diluting the drug product with water, buffer, ethanol or methanol to a suitable concentration). Comparison with the absorbance of a standard solution of 17-β-estradiol 3-phosphate disodium salt and the 17-α-methyltestosterone 3-phosphate salt is performed. Alternatively, HPLC assay may be used to compare the absorbance of the paraben-containing placebo versus the absorbance of the active drug formulation.

In yet an alternate embodiment, it is contemplated that the composition of said invention be free of any preservative compounds and said invention be provided to patient in a sterile single or similar package allowing no more than 7 days of use before the patient discards the package.

In another alternate embodiment, it is contemplated that the present invention utilizes an ocular insert means of delivering the combination steroids directly to the ocular surface and conjunctiva. Such delivery systems are well known in the art and are exemplified by the disclosure of U.S. Pat. No. 4,478,818, and hereby incorporated by reference.

In yet another alternate embodiment, it is contemplated that the present invention utilizes a thermosetting gel with a low sol-gel transition temperature as a method of delivering the combination steroid ingredients directly to the ocular surface and conjunctiva. Such delivery systems are well known in the art and are exemplified by the disclosure of U.S. Pat. No. 4,474,571, and also hereby incorporated by reference.

In yet another alternate embodiment, it is contemplated that the present invention utilizes the combination steroids as an encapsulated agent for introduction into the suprachoroid of the eye for therapeutic purposes. The administration of the steroids can be controlled and maintained for long periods of time, while ensuring the substantial absence of significant concentrations of steroids outside the site of administration. Examples of such materials and techniques are shown in the art (U.S. Pat. No. 4,853,224, U.S. Pat. No. 4,997,652, U.S. Pat. No. 5,164,188, U.S. Pat. No. 5,443,505, U.S. Pat. No. 5,766,242) and are hereby incorporated by reference.

The quality control procedures are also the same as for the drug product described above with the exception that the ultraviolet absorbance at 280 nanometers of the placebo solution when diluted to the same concentration as the drug product will fail to indicate the presence of steroids in representative samples of the placebo solution.

### EXAMPLE 1

A liposome delivery vehicle is shown in the table below.

TABLE V
A Liposome Delivery Vehicle

| Ingredient | Amount (w/w %) |
|---|---|
| 17-β-estradiol | Desired amount |
| 17-α-methyl-testosterone | Desired amount |
| Phosphotidylcholine | 3.0 |
| Phosphotidylserine | 3.0 |
| Carbomer (N.F.) | q.s. |
| Propylene glycol | 6.0 |
| C₁₂₋₁₅ benzoate | 2.0 |
| Emulsifying wax | 2.0 |
| Aminomethyl propanol | q.s. |
| Preservative (optional) | 1.0 |
| Purified water (U.S.P.) | q.s. |

Disperse the carbomer (a polymer of acrylic acid used in pharmaceutical preparations) in a portion of the purified water and heat to about 70° C. Add the steroids in the emulsifying wax, C₁₂₋₁₅ benzoate, both phospholipid derivatives and propylene glycol to about 70° C. Cool the solution to about 40° C. and adjust the pH of the solution to about pH 6.0 with the aminomethyl propanol. Add the preservative (if any) and add additional purified water to the final desired volume. While warm, filter under pressure through 0.2 μM membrane filter to form a sterile solution. Note that this method is described for example purposes and is not intended to show the only method that is possible.

The contemplated treatment regime of this invention requires application of a pharmacologically effective amount of active ingredients as described, in unit dose form, to an eye presenting elevated intraocular pressure, maintaining an effective unit dose amount applied for a sufficient period of time to assure that a lowered intraocular pressure is maintained. For example, intraocular pressure six months after a constant physiologic or normal intraocular pressure is obtained may be used as a benchmark to ensure that the affected eye is maintained at physiologic or normal eye pressure. The amount of active ingredients administered to the eye may be decreased after this point, either by decreasing the amount of active ingredient or decreasing the frequency of application to the eye. During this period of adjustment of dosage, the eye should be monitored for any change in intraocular pressure or visual field.

It will be apparent to those skilled in the art that various modifications and variations can be made to the present invention without departing from the spirit or scope of the invention. Thus, it is intended that the present invention cover the modifications and variations of this invention provided they come within the scope of the appended claims and their equivalents. Accordingly, the invention is not limited by the embodiments described above which are presented as examples only, but can be modified in various ways within the scope of protection defined by the appended patent claims.

We claim:

1. An eye drop composition for the treatment of age-related maculopathy, age-releted macular degeneration, ocular hypertension, glaucoma or other eye disease involving increased intraocular pressure comprising anabolic androgenic agents and 17-β-estradiol, or its esters, in a lipid vehicle or 17-β-estradiol-3-phosphate, or other esters of 17-β-estradiol and androgenic agents in an aqueous vehicle, and having a pH of between about pH 6 to pH 8.

2. The composition of claim 1, wherein the concentration of 17-β-estradiol, or its esters, in the lipid vehicle or 17-β-estradiol-3-phosphate, or other esters of 17-β-estradiol in the aqueous vehicle is 0.001% to 1% (w/w)

3. The composition of claim 1, wherein the concentration of anabolic androgenic agent or its esters in the lipid vehicle or the aqueous solution is 0.001% to 1% (w/w)

4. The composition of claim 1, wherein the anabolic androgenic agent is one selected from the group consisting of 17-α-methyl-testosterone, oxandrolone, norethandrolone, bolasterone, methandrostenolone, oxymetholone, and dihydrotestosterone, their esters or phosphate derivatives.

5. The composition of claim 1, wherein the anabolic androgenic agent is 17-α-methyl-3-phosphate-testosterone.

6. A method for decreasing elevated intra-ocular pressure, or treating age-related maculopathy or age-related macular degeneration comprising administering to an eye in need of such treatment, an eye drop composition comprising as active ingredients, an effective amount of anabolic androgenic agents and 17-β-estradiol, or its esters, in a lipid vehicle or an anabolic agent and 17-β-estradiol-3-phosphate, or other esters of 17-β-estradiol, in an aqueous vehicle, and having a pH of between about pH 6 to pH 8.

7. The method of claim 6, wherein the concentration of 17-β-estradiol, or its esters, in a lipid vehicle or 17-β-estradiol, 17-β-estradiol-3-phosphate, or other esters of 17-β-estradiol, in an aqueous vehicle is 0.001% to 1% (w/w)

8. The method of claim 6 wherein the concentration of anabolic androgenic agent or its esters in a lipid vehicle or an aqueous solution is 0.001% to 1% (w/w)

9. The method of claim 6, wherein the anabolic androgenic agent is one selected from the group consisting of 17-α-methyl-testosterone, oxandrolone, norethandrolone, bolasterone, methandrostenolone, oxymetholone, and dihydrotestosterone.

10. The method of claim 6, wherein the anabolic androgenic agent is 17-α-methyl-3-phosphate-testosterone.

11. The method of claim 6, wherein said 17-β-estradiol or its derivatives and an androgenic agent or derivatives are dissolved in an articficial tear-like vehicle.

12. The method of claim 6, wherein said 17-β-estradiol or its derivatives and an androgenic agent or derivatives are dissolved in a delivery vehicle comprising on a w/w basis:

| | |
|---|---|
| Dibasic sodium phosphate, USP | 0.05-1.0%; |
| Sodium Chloride, USP | 0.2-0.9%; |
| Edetate disodium, USP | 0.05-1.0%; |
| Povidone, USP | 0.05-2.0%; |
| Poloxamer NF | 0.001-0.05%; |
| Polyethylene glycol | 0.05-1.0%; |
| Hydroxyethyl Cellulose NF | 0.05-1.0%; |
| Purified water, USP | q.s to 100%; and |
| HCl or NaOH | to adjust pH to pH 6-8. |

13. The method of claim 6, wherein said 17-β-estradiol or its derivatives and an androgenic agent or derivatives are dissolved or suspended in a vehicle comprising on a w/w basis:

| | |
|---|---|
| Dibasic sodium phosphate, USP | 0.3%; |
| Sodium Chloride, USP | 0.6%; |
| Edetate disodium, USP | 0.1%; |
| Povidone K-17, USP | 0.37%; |
| Poloxamer NF, | 0.004%; |
| Polyethlyene glycol | 0.12%; |
| Hydroxyethyl Cellulose NF | 0.2%; |
| Purified water, USP, | q.s to 100%; and |
| HCl or NaOH | to adjust pH to pH 6-8. |

14. The method of claim 6, wherein the delivery vehicle further comprises a preservative.

15. The method of claim 14, wherein the preservative is at least one selected from the group consisting of methlyparaben, propylparaben, benzalkonium chloride and phenoxyethanol.
